(19) United States
(12) Patent Application Publication
(10) Pub. No.: US 2023/0123884 A1
(43) Pub. Date: Apr. 20, 2023

(54) BUBBLE TRAP LEVEL CONTROL USING NON-CONTACTING CAPACITANCE PROBES

(71) Applicant: Innovative Engineering Systems, Inc., Bakersfield, CA (US)

(72) Inventor: Dan Klees, Bakersfield, CA (US)

(21) Appl. No.: 17/967,104

(22) Filed: Oct. 17, 2022

Related U.S. Application Data
(60) Provisional application No. 63/257,235, filed on Oct. 19, 2021.

(51) Int. Cl. A61M 1/36; G01F 25/20

(52) U.S. Cl. CPC A61M 1/363; G01F 25/20; G01F 23/263

(57) ABSTRACT

Systems and methods for non-contact sensing of the level of process fluid within a hygienic bubble trap are contemplated. In particular, it is contemplated that through the use of one or more capacitance probes having their sensor tips placed adjacent to the vessel wall of a hygienic bubble trap, ideally perpendicularly thereto, the fluid level of the process fluid within the hygienic bubble trap may be measured via the circuit of the capacitance probe detecting variances in the electrical field which extends through the vessel wall, as a result of changes in the dielectric as the process fluid rises and falls. In this fashion, the probe is not wetted and is never placed into physical contact with the process fluid, and thus does not need to undergo any clean-in-place or sterilize-in-place processes, greatly simplifying ease of use.

### CROSS-REFERENCE TO RELATED APPLICATIONS

This patent application claims the benefit and priority of U.S. Provisional Patent Application No. 63/257,235, entitled BUBBLE TRAP LEVEL CONTROL USING NON-CONTACTING CAPACITANCE PROBES, filed on Oct. 19, 2021, the disclosure of which is incorporated by reference herein in its entirety as part of the present application.

### STATEMENT RE: FEDERALLY SPONSORED RESEARCH/DEVELOPMENT

Not Applicable

### BACKGROUND

### 1. Technical Field

The present disclosure relates generally to the fields of hygienic bubble traps. More particularly, the present disclosure relates to systems and methods for controlling fluid level within a hygienic bubble trap within a fluid level range.

A hygienic bubble trap, or just a bubble trap, is device that is used in fluidic flow systems to remove unwanted air and gas bubbles from the fluid phase. Hygienic bubble traps are used extensively in downstream biopharmaceutical purification processes such as chromatography. The presence of gas bubbles in fluidic flow operations, such as in process fluids and buffer solutions, can cause significant problems within the system because the bubbles may unexpectedly change the intrinsic physical properties of the fluidic environment, such as sudden shear force variations, or abrupt pressure changes. Furthermore, in a chromatography system, the presence of bubbles can affect the operation of a resin bed by impeding flow through the resin column, as well as limiting contact with the resin. The result may be a decrease in separation efficiency and resolution.

Hygienic bubble traps operate to remove unwanted gas or air bubbles in process fluids by allowing the separation of the gas bubbles from the fluid phase. In a bubble trap, gas bubbles rise to the top while fluid remains at the bottom, due to buoyant forces on the gas bubbles. The design of the bubble trap and its proper operation is critical for the efficient removal of gas bubbles from the process fluid, as well as to prevent bubbles from being drawn into the hygienic bubble trap fluid outlet.

One of the critical factors in efficient operation of a hygienic bubble trap is its working volume. A typical bubble trap may efficiently operate, for example, based upon a residence time of the fluid of approximately 15 seconds within the bubble trap. Therefore, the size of the bubble trap (which may be seen to generally govern its working volume) may principally govern how much fluid may be processed therein at any given time, as a function of its internal working volume (usually between 60% to 80% of the total internal volume). For example, if the flow rate of process fluid within a fluidic system is 4 liters/minute and the bubble trap residence time is 15 seconds, then the bubble trap used should be sized to have an internal working volume of 1 liter or greater.

As may be appreciated, the interrelationship between the internal working volume and the total internal volume of the bubble trap is critical to the efficient separation of gas bubbles from the process fluid. This relationship can be generally conceptualized as the fluid level within the bubble trap. If the fluid level within the bubble trap is too low, then the buoyant forces upon of the gas bubbles will not have a residence time in the bubble trap to permit the gas bubbles to rise above the fluid before being drawn into the bubble trap fluid outlet. Likewise, if the fluid level within the bubble trap is too high, then fluid may be drawn into the gas vent line, which may result in issues with cleaning and sterilization, and may result in batch contamination or bioburden.

Current systems for controlling the fluid level within a hygienic bubble trap suffer from a number of drawbacks, as will be more fully explained herein.

### BRIEF SUMMARY

To solve these and other problems, systems and methods for non-contact sensing of the level of process fluid within a hygienic bubble trap are contemplated. In particular, it is contemplated that through the use of one or more capacitance probes having their sensor tips placed adjacent to the vessel non-conductive wall of a hygienic bubble trap, ideally perpendicularly thereto, the bubble trap vessel non-conductive wall, fluid within the hygienic bubble trap may be sensed via the circuit of the capacitance probe detecting variances in the electrical field which extends through the vessel wall, as a result of changes in the sensed dielectric as the process fluid rises and falls. In this fashion, the probe is not wetted and is never placed into physical contact with the process fluid, and thus does not need to undergo any clean-in-place or sterilize-in-place processes, greatly simplifying ease of use.

According to one embodiment of the present disclosure, a method for non-contact sensing of the level of process fluid within a hygienic bubble trap having an internal volume and a vessel wall is contemplated, the method comprising the steps of providing one or more capacitance probes comprising a sensor tip and an oscillator circuit, and positioning the sensor tip external to the vessel wall of the hygienic bubble trap and not in direct contact with the process fluid, activating the oscillator circuit to generate an electrical field at the sensor tip, the electrical field being operative to penetrate the low dielectric vessel wall, and determining the presence of a change in the level of the process fluid within the hygienic bubble trap via detection at one or more of the oscillator circuits of one or more of the capacitance probes of one or more of: a voltage drop measured across a reference resistor, a change in measured resonant frequency, or combinations thereof.

According to additional embodiments, it is contemplated that the capacitance probes are further operative to determine whether the fluid level of the process fluid within the hygienic bubble trap is within one or more predefined fluid level states. At least of the one or more predefined level states may comprise a low-level state, and in response to the determination by the one or more capacitance probes that the fluid level of the process fluid within the hygienic bubble trap is within the low-level state, the hygienic bubble trap is configured to vent headspace gas.

It is further contemplated that among the capacitance probes, there may be a high-level capacitance probe and a low-level capacitance probe. The high-level capacitance probe may be operative to determine whether the fluid level of the process fluid within the hygienic bubble trap is within a high-level state, and the low-level capacitance probe may be operative to determine whether the fluid level of the process fluid within the hygienic bubble trap is within a low-level state. In response to a determination by the low-level capacitance probe that the fluid level of the process fluid within the hygienic bubble trap is within a low-level state, the hygienic bubble trap is configured to vent headspace gas, thereby increasing the fluid level within the bubble trap. Likewise, in response to a determination by the high-level capacitance probe that the fluid level is of the process fluid within the hygienic bubble trap is within a high-level state, the hygienic bubble trap is configured to cease venting headspace gas.

It is further contemplated that the vessel wall of the hygienic bubble trap may be comprised of borosilicate glass or any other non-conductive material such as soda-lime glass, plastics and polymers, and that each sensor tip of the one or more capacitance probes are mounted about perpendicular to the vessel wall.

Furthermore, a system for non-contact sensing the level of process fluid within a hygienic bubble trap having an internal volume and a vessel wall is also contemplated, the system comprising one or more capacitance probes comprising a sensor tip and an oscillator circuit, the one or more capacitance probes being positioned external to the vessel wall of a hygienic bubble trap and not in direct contact with process fluid therein, the oscillator circuit being operative to generate an electrical field at the sensor tip, the electrical field being operative to penetrate the vessel wall, and a controller for evaluating the presence of a change in the level of the process fluid within the hygienic bubble trap via detection at one or more of the oscillator circuits of one or more of the capacitance probes of one or more of: a voltage drop measured across a reference resistor, a change in measured resonant frequency, or combinations thereof, wherein the system is operative to determine the presence of a change in the level of the process fluid within the hygienic bubble trap via detection at one or more oscillator circuits of or more of the capacitance probes of one or more of: a voltage drop measured across a reference resistor, a change in measured resonant frequency, or combinations thereof. This system is contemplated as being capable of all of the methods described above.

### BRIEF DESCRIPTION OF THE DRAWINGS

These and other features and advantages of the various embodiments disclosed herein are better understood with respect to the following descriptions and drawings, in which:

FIG. 1 is an illustration of a conventional hygienic bubble trap;

FIG. 2 is a schematic diagram illustrating the components of a conventional hygienic bubble trap;

FIG. 3 is a simplified diagram of a conventional capacitance sensor;

FIG. 4 is a schematic diagram of the circuit of a conventional capacitance sensor;

FIG. 5 is an illustration of a prior art capacitance probe scheme wherein the probe directly contacts the process fluid;

FIG. 6 is an illustration of a circuit of the prior art capacitance probe scheme shown in FIG. 5;

FIG. 7 is an illustration of a capacitance probe scheme according to the present disclosure where the capacitance probe does not directly contact the process fluid;

FIG. 8 is a schematic diagram of a simplified circuit of a capacitance probe according to the present disclosure; and

FIG. 9 is a process diagram showing the operation of a dual noncontact capacitance probe point level sensing system according to the present disclosure.

### DETAILED DESCRIPTION

As is contemplated by the disclosure herein, systems and methods for non-contact sensing of the level of process fluid within a hygienic bubble trap are contemplated. In particular, it is contemplated that through the use of one or more capacitance probes having their sensor tips placed adjacent to the vessel wall outside of a hygienic bubble trap, ideally perpendicularly thereto, the fluid level of the process fluid within the hygienic bubble trap may be measured via the circuit of the capacitance probe detecting variances in the electrical field which extends through the vessel wall, as a result of changes in the sensed dielectric as the process fluid rises and falls. In this fashion, the probe is not wetted and is never placed into physical contact with the process fluid, and thus does not need to undergo any clean-in-place or sterilize-in-place processes, greatly simplifying ease of use.

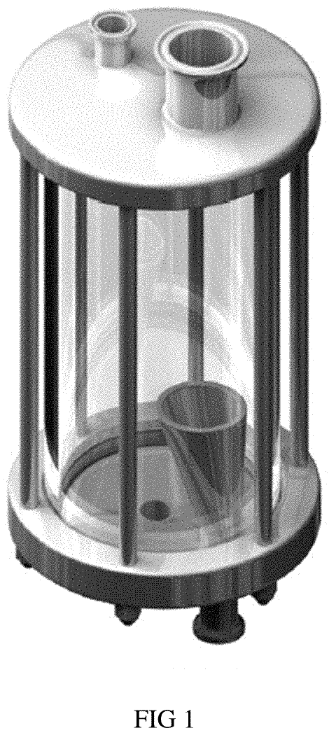

Turning now to FIG. 1, a conventional hygienic bubble trap is illustrated. As may be seen, a conventional bubble trap typically includes at least a fluid inlet having an inlet eccentric nozzle, a fluid outlet, vessel walls, and a gas vent. With respect to the vessel walls, that may comprise borosilicate glass or any other non-conductive material such as soda-lime glass, plastics and polymers known in the art.

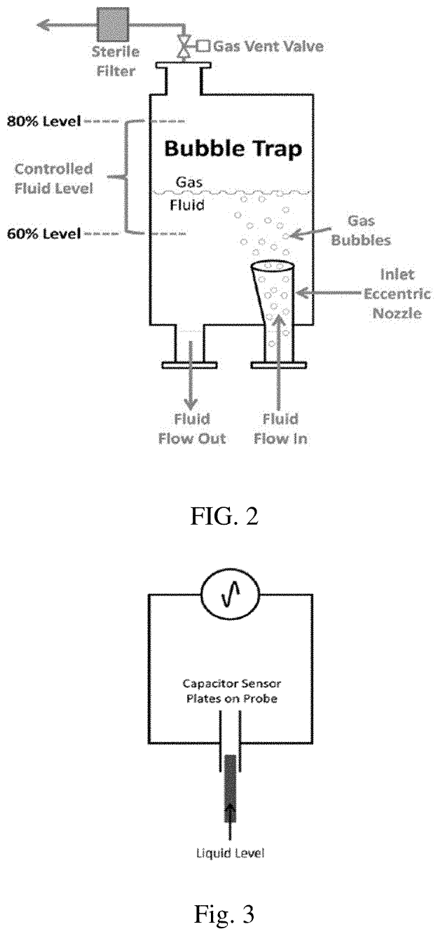

Turning now to FIG. 2, a schematic diagram of a conventional hygienic bubble trap is illustrated. As may be seen, during typical operation of a bubble trap, it is not generally required to control the exact fluid level within the bubble trap, but rather it is generally necessary to control the fluid level to within a particular range. This is because the fluid level within a bubble trap does not typically remain constant during the operation of many types of fluidic systems. For example, in a chromatographic purification system, the process tubing, purification equipment, and bubble trap begins empty. As fluid enters the hygienic bubble trap, the gas line vent valve will begin closed, and fluid inflow into the bubble trap will exceed outflow until the pressure of the fluid and of the gas headspace above the fluid will reach equilibrium. At this equilibrium point, the level of the fluid within the bubble trap will typically remain constant due to fluid inflow being equal to fluid outflow.

Thereafter, the level of the fluid will only change if there is an imbalance between inlet pressure, outlet pressure, and gas headspace pressure. If the headspace pressure remains constant, fluid flow in will generally equal fluid flow out, and the fluid level will remain constant. However, as the bubble trap operates and gas bubbles are separated from the fluid due to buoyant force and migrate to the headspace, the volume of the gas within the gas headspace will increase. Accordingly, the fluid level equilibrium point will begin to decrease to a lower level. In order to remedy this, the gas vent will be opened, venting some volume of the gas within the headspace, causing a rise in the fluid level.

In conventional prior art devices, point level sensors are typically utilized in order to keep the fluid level between 60% and 80% of the maximum bubble trap height. According to such conventional embodiments of prior art bubble traps, when the level of the fluid reaches the point where it is detected by point level sensor positioned at the 60% point, the gas vent is configured to be opened, and thereafter once the level of the fluid is detected by the point level sensor positioned at the 80% point, the gas vent is configured to close. Other ranges of control are also equally possible. Likewise, continuous level probes have also been utilized in connection with a PID control algorithm in order to modulate the opening and closing of the gas vent control valve, thereby controlling the level of the fluid within the bubble trap. However, continuous level sensing and control is substantially more expensive than point level control and is much more suitable for applications requiring point level control, as opposed to the general range of 60% to 80% of the total volume.

Likewise, multiple prior art technologies have been utilized in order to previously measure the fluid level within a hygienic bubble trap. Generally, these technologies can be divided into two categories: (1) process contact technologies, and (2) non-process contact technologies.

Process contact technologies have included conductivity, capacitance, differential pressure, and RADAR (guided wave as well as through air). However, each of these technologies has various drawbacks. In particular, since the level sensor is either in direct contact with the fluid or the gas in the bubble trap, the sensors must meet certain conditions and potential regulatory measures with regards to cleanability, sterilizability, potential sensor wetted parts requirements, sensor surface finish requirements, hold-up volumes, potential sensor extractables and leachables, and potential sensor chemical reactivity with the purification process. The risk of using a direct process contact technology in a hygienic process must be evaluated and taken not account. On the other hand, if a level measuring technology does not involve physical contact with the process fluid or the gas, the above requirements do not need to be evaluated or a risk assessment conducted.

Certain non-process contact technologies have been successfully used to measure hygienic bubble trap levels. In non-contact application, sensors are used to sense the presence or absence of fluids in a bubble trap through the bubble trap vessel wall (typically a glass cylinder) and may be physically isolated from the hygienic process thereby. Suh non-process contact sensor technologies have included optical technologies such as measurements of transmission, refraction, and reflection of light and lasers.

Turning now to FIG. 3, an illustration of a conventional capacitive level sensor is shown. A conventional capacitive level sensor consists of two conductive electrodes (plates) forming a sensing capacitor with insulating material separating the electrodes. As an alternating current is applied to the circuit connecting the two electrodes, the capacitor stores a quantity electrostatic energy in the electric field between the capacitor's electrodes, a measure known as capacitance. The capacitance increases as the volume of fluid between the electrode increases.

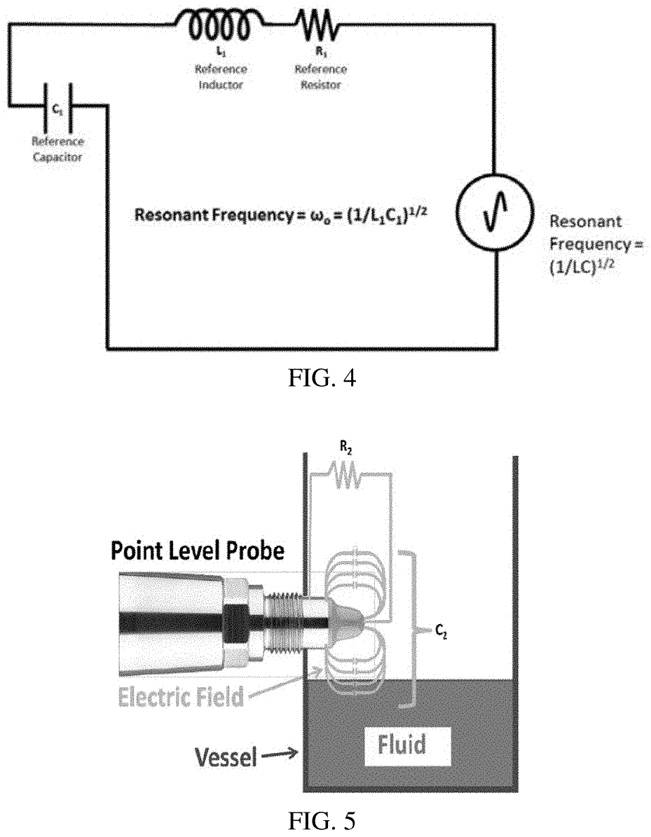

Turning now to FIG. 4, a schematic of an electrical circuit for a conventional capacitive level sensor is shown. As may be seen, capacitive level sensors work by sensing capacitance with reference to the dielectric constant of the material being measured and the voltage being used to complete the circuit. This method of level measurement is sometimes called RF level, since radio frequencies are applied to the capacitor circuit. These capacitive measures are used to infer fluid levels in a vessel. Because the dielectric of fluid (e.g., water) is typically greater than that of gases (e.g., atmosphere), as the fluid level rises, the detected capacitance will increase, because more dielectric material will occupy the electric field between the capacitor's electrodes. Higher levels of dielectric material result in greater capacitance, meaning that the fluid level can be inferred by measuring electrical capacitance. A change in the fluid's dielectric properties will also be seen as an apparent change in the fluid level. Thus, a capacitance level sensor of this type will need to typically be calibrated using a fluid with approximately the same dielectric constant as the process fluid to be measured.

Point level capacitance sensors may also operate on these same principals. However, a point level capacitor may simply have the capacitance charge stored between the electrodes be compared to a preconfigured target capacitance, and a switch contact enabled when the capacitance exceeds a predefined target.

Turning now to FIG. 5, an illustration of a conventional point level capacitance probe as described above is shown. As may be seen, the probe will be in contact with the process fluid and will become wetted by the process. The electric circuit will need to take into account the effect of changing process fluid dielectric and resistance as the sensor becomes wetted.

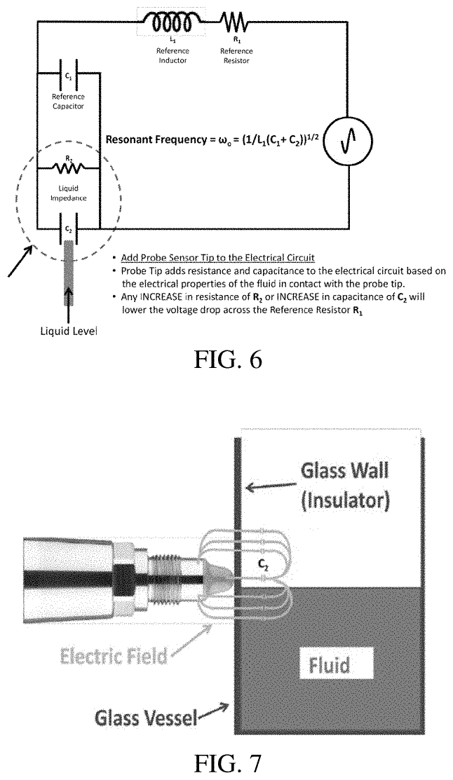

Turning now to FIG. 6, an illustration is shown of an electrical circuit for the capacitance point level probe that is in direct contact with the process fluid, as shown in FIG. 5. As may be seen, the probe tip itself adds resistance and capacitance to the circuit based on the properties of the fluid in contact with the probe tip, and either an increase in the resistance of the liquid (R₂) or an increase in capacitance between the electrodes (C₂) will result in a voltage drop across a reference resistor (R₁).

Turning now to FIG. 7, an illustration of an embodiment of the presently contemplated scheme for non-contacting capacitance level probe operation is shown. As may be seen, the capacitance level probe is isolated from the process fluid by the glass wall of the bubble trap. The sensor probe never makes any physical contact with the process fluid. Rather, the electrical field generated by the frequency oscillator penetrates through low dielectric solids such as glass or plastics. Glass is an insulator, and has a relatively low dielectric of approximately 5, as well as very low conductivity (high resistance), compared to most buffer solutions which have a dielectric constant over 50.

As may be seen, the capacitance level sensor probe will not be in direct physical contact with the process fluid, but rather will be isolated from the process fluid by the vessel wall of the bubble trap (typically a glass cylinder). Instead, the electric RF field generated by the oscillator will penetrate through the glass wall insulator.

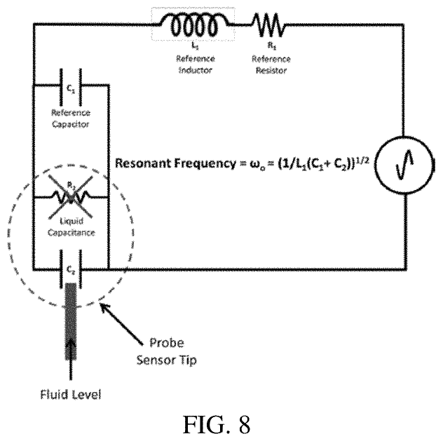

Turning now to FIG. 8, the simplified electrical circuit for a non-contact capacitance level sensor as presently contemplated is shown. As may be seen, as the electrical resistance of the vessel wall will be extremely high, the resistance R₂ will have minimal effect on the electrical circuit and may be entirely ignored for purposes of determining fluid level. Rather, it may be appreciated that the resistance R₂ may be seen to only be important in applications where the level sensor probe tip is in direct contact with the process fluid. As such, the resistance R₂ value can thus be ignored entirely, which may simplify operations of the probe greatly.

Because the capacitance values of the reference capacitor (C₁) and the capacitance value of the probe sensor tip (C₂) are in parallel, and the total capacitance of the circuit will be C₁+C₂. As such, any change in the sensor capacitance C₂ will result in a change in the voltage drop across the reference resistor R₁. Therefore, in order to determine that a change in the fluid level has occurred, the sensor only has to compare either the change in voltage drop across R₁, or the change in the resonant frequency ω₀, which can be measured by the equation (1/(L₁*(C₁+C₂))\hat{ }(½), where L₁ is the induction across a reference inductor, due to the fact that the resonant frequency will vary as C₂ varies in capacitance.

According to one exemplary embodiment, two-point level switches were installed external to the vessel wall of a bubble trap, one as a low-level sensor, and one as a high-level sensor. During normal operation of the bubble trap, the fluid level should be between the low-level probe and the high-level probe. Should the fluid level drop below the low-level sensor, the gas vent will be configured to be opened, permitting gas to be vented through a sterile filter, resulting in an increase in fluid level due to a drop in the volume of the headspace gas, as described in detail above. As the gas is vented, the fluid level will increase until it reaches the high-level sensor, which will then sense the fluid level and cause the gas vent to close. The point level switches were mounted to support rods of the hygienic bubble trap via boss clamps and were isolated from the process fluid by the presence of the borosilicate glass wall.

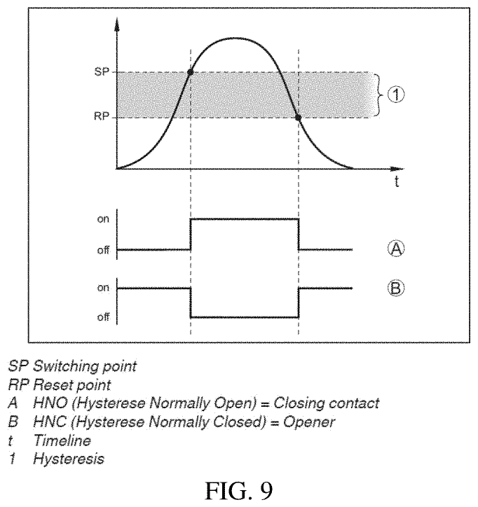

Turning now to FIG. 9, a process diagram showing the operation of a dual noncontact capacitance probe point level sensing system according to one embodiment of the present disclosure is shown. As illustrated, when the fluid level exceeds the acceptable boundaries (hysteresis region 1) the low-level sensor triggers a switching point, which causes an electrical contact to result in actuation opening the gas vent. Thereafter, as the fluid level rises due to the outflow of headspace gas, the threshold of the high-level sensor is reached, resulting in the triggering of the reset point, causing the electrical contact to open and triggering an actuating closing of the gas vent. According to this embodiment, the difference between the probe sensing air and the probe sensing a high dielectric fluid such as water was determined to be a difference in the measured resonant frequency of 4%, with a resonant frequency reading of 83% when distilled water (a high dielectric fluid) was sensed, and a reading of 87% when air (a low dielectric fluid) was sensed. Thus, the resonant frequency increased when a low dielectric gas was sensed and decreased when a high dielectric process fluid was sensed.

Important factors in allowing capacitance determination and improving repeatability of the system include the thickness and dielectric of the vessel wall of the bubble trap, the mounting angle of the probe with respect to the vessel wall, and the dielectric of the process fluid. Proper optimization of these parameters has permitted a repeatability of point level determination to up to ⅛ of an inch. However, even without substantial optimization, a repeatability of ¼ of an inch is readily achievable. Likewise, the presence of external interference with the electric field of the probe has a minimal effect. The mounting of the probe was found to be important with respect to repeatability, and it was found that the probe should be mounted as close as possible to perpendicular to the vessel wall, and ideally in contact with the vessel wall.

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the exemplary embodiments.

What is claimed is:

1. A method for non-contact sensing of the level of a process fluid within a hygienic bubble trap having an internal volume and a vessel wall, the method comprising the steps of:
providing one or more capacitance probes comprising a sensor tip and an oscillator circuit, and positioning the sensor tip external to the vessel wall of the hygienic bubble trap and not in direct contact with the process fluid;
activating the oscillator circuit to generate an electrical field at the sensor tip, the electrical field being operative to penetrate the vessel wall; and
determining the presence of a change in the level of the process fluid within the hygienic bubble trap via detection at one or more of the oscillator circuits of one or more of the capacitance probes of one or more of: a voltage drop measured across a reference resistor, a change in measured resonant frequency, or combinations thereof.

2. The method of claim 1, wherein the capacitance probes are further operative to determine whether the fluid level of the process fluid within the hygienic bubble trap is within one or more predefined fluid level states.

3. The method of claim 2, wherein at least of the one or more predefined level states comprises a low-level state.

4. The method of claim 3, wherein in response to the determination by the one or more capacitance probes that the fluid level of the process fluid within the hygienic bubble trap is within the low-level state, the hygienic bubble trap is configured to vent headspace gas.

5. The method of claim 1, wherein the capacitance probes comprise a high-level capacitance probe and a low-level capacitance probe.

6. The method of claim 5, wherein the high-level capacitance probe is operative to determine whether the fluid level of the process fluid within the hygienic bubble trap is within a high-level state, and wherein the low-level capacitance probe is operative to determine whether the fluid level of the process fluid within the hygienic bubble trap is within a low-level state.

7. The method of claim 6, wherein in response to a determination by the low-level capacitance probe that the fluid level is of the process fluid within the hygienic bubble trap is within the low-level state, the hygienic bubble trap is configured to vent headspace gas.

8. The method of claim 6, wherein in response to a determination by the high-level capacitance probe that the fluid level is of the process fluid within the hygienic bubble trap is within the high-level state, the hygienic bubble trap is configured to cease venting headspace gas.

9. The method of claim 1, wherein the vessel wall of the hygienic bubble trap comprises borosilicate glass and has a dielectric constant of about 5.

10. The method of claim 1, wherein each sensor tip of the one or more capacitance probes are mounted about perpendicular to the vessel wall.

11. A system for non-contact sensing the level of a process fluid within a hygienic bubble trap having an internal volume and a vessel wall, the system comprising:
one or more capacitance probes comprising a sensor tip and an oscillator circuit, the one or more capacitance probes being positioned external to the vessel wall of the hygienic bubble trap and not in direct contact with the process fluid therein, the oscillator circuit being operative to generate an electrical field at the sensor tip, the electrical field being operative to penetrate the vessel wall; and
a controller for evaluating the presence of a change in the level of the process fluid within the hygienic bubble trap via detection at one or more of the oscillator circuits of one or more of the capacitance probes of one or more of: a voltage drop measured across a reference resistor, a change in measured resonant frequency, or combinations thereof;
wherein the system is operative to determine the presence of a change in the level of the process fluid within the hygienic bubble trap via detection at one or more oscillator circuits of or more of the capacitance probes of one or more of: a voltage drop measured across a reference resistor, a change in measured resonant frequency, or combinations thereof.

12. The system of claim 11, wherein the capacitance probes are further operative to determine whether the fluid level of the process fluid within the hygienic bubble trap is within one or more predefined fluid level states.

13. The system of claim 12, wherein at least of the one or more predefined level states comprises a low-level state.

14. The system of claim 13, wherein in response to the determination by the one or more capacitance probes that the fluid level of the process fluid within the hygienic bubble trap is within the low-level state, the hygienic bubble trap is configured to vent headspace gas.

15. The system of claim 11, wherein the capacitance probes comprise a high-level capacitance probe and a low-level capacitance probe.

16. The system of claim 15, wherein the high-level capacitance probe is operative to determine whether the fluid level of the process fluid within the hygienic bubble trap is within a high-level state, and wherein the low-level capacitance probe is operative to determine whether the fluid level of the process fluid within the hygienic bubble trap is within a low-level state.

17. The system of claim 16, wherein in response to a determination by the low-level capacitance probe that the fluid level is of the process fluid within the hygienic bubble trap is within the low-level state, the hygienic bubble trap is configured to vent headspace gas.

18. The system of claim 16, wherein in response to a determination by the high-level capacitance probe that the fluid level is of the process fluid within the hygienic bubble trap is within the high-level state, the hygienic bubble trap is configured to cease venting headspace gas.

19. The system of claim 11, wherein the vessel wall of the hygienic bubble trap comprises borosilicate glass and has a dielectric constant of about 5.

20. The system of claim 11, wherein each sensor tip of the one or more capacitance probes are mounted about perpendicular to the vessel wall.
